(19) United States
(12) Patent Application Publication
(10) Pub. No.: US 2026/0108372 A1
(43) Pub. Date: Apr. 23, 2026

(54) INTRALUMINAL DEVICE AND METHOD OF APPLYING STRESS TO BARORECEPTORS IN THE GASTRO-INTESTINAL TRACT

(71) Applicant: BFKW, LLC, Grand Rapids, MI (US)

(72) Inventors: Paul R. Kemmeter, Lowell, MI (US); Randal S. Baker, Grand Rapids, MI (US); Frederick J. Walburn, Grand Rapids, MI (US)

(21) Appl. No.: 18/880,351

(22) PCT Filed: Jun. 4, 2024
(86) PCT No.: PCT/IB2024/055458

§ 371 Date: Dec. 31, 2024

Related U.S. Application Data
(60) Provisional application No. 63/615,605, filed on Dec. 28, 2023.
(60) Provisional application No. 63/552,928, filed on Feb. 13, 2024.

(51) Int. Cl. A61F 5/00

(52) U.S. Cl. CPC A61F 5/0036

(57) ABSTRACT

A method of stimulating baro receptors at an inner surface of the gastro-intestinal tract of a patient and an intraluminal device includes fixing the intraluminal device to a portion of the inner surface of the gastro-intestinal tract with a magnetic fastener. The fixing results in applying stress with a baro receptor stimulating surface to the portion of the inner surface of the gastro-intestinal tract. The magnetic fastener includes a magnet assembly and an attractor assembly. A first assembly, including either the magnet assembly or attractor assembly, is positioned against the inner surface or an outer surface at the portion gastro-intestinal tract. A second assembly, including the wall and the other of the magnet assembly or attractor assembly, is positioned against the portion of the inner surface of the gastro-intestinal tract. Magnetic attraction between the magnet and attractor assemblies applies stress with the baro receptor stimulating surface to stimulate baro receptors.

### BACKGROUND OF THE INVENTION

The present invention is directed to a method and system for applying stress to baroreceptors concentrated in portions of the gastro-intestinal tract and, in particular, to deploying and fixing an intraluminal device in a manner that applies stress to stimulate barro receptors at the inner wall of the gastro-intestinal tract.

### SUMMARY OF THE INVENTION

The invention is illustrated for use with applying stress to the cardiac portion of the stomach. The positioning of the intraluminal device against the cardiac portion of the stomach simulates a sensation of fullness in the patient's stomach in the absence of food or supplement fullness from the presence of food, using a variety of techniques.

The invention provides a minimally invasive technique for securely affixing an intraluminal device. The technique facilitates the rapid and simplified exchange of the intraluminal device for the purpose of modifying the level of stimulation of the baro receptors at the cardiac portion of the stomach. This provides an effective manner to titrate level of stimulation to the current condition of the patient. In this manner, the intraluminal device has the potential to remain in place throughout all, or an extended portion, of the life of the patient.

A method of stimulating baro receptors at an inner surface of the gastro-intestinal tract of a patient and an intraluminal device, according to an aspect of the invention, includes fixing an intraluminal device to a portion of the inner surface of the gastro-intestinal tract with a magnetic fastener. The intraluminal device includes a wall defining a baro receptor stimulating surface. The baro receptor stimulating surface and the portion of inner surface of the gastro-intestinal tract are conformable with each other. The fixing results in applying stress with the baro receptor stimulating surface to the portion of the inner surface of the gastro-intestinal tract. The magnetic fastener includes a magnet assembly and an attractor assembly. A first assembly, including either the magnet assembly or attractor assembly, is positioned against the inner surface or an outer surface at the portion gastro-intestinal tract. A second assembly, including the wall and the other of the magnet assembly or attractor assembly, is positioned against the portion of the inner surface of the gastro-intestinal tract. Magnetic attraction between the magnet and attractor assemblies applies stress with the baro receptor stimulating surface to stimulate baro receptors.

The first assembly may be deployed to the abdominal cavity and positioned against the outer surface of the gastro-intestinal tract. Alternatively, the first assembly is deployed to the interior of the gastro-intestinal tract and fixed to the portion of the inner surface of the gastro-intestinal tract. The second assembly may have an open portion of the baro receptor stimulating surface and the abdominal assembly magnetically generally aligns the open portion of the surface with the gastro-intestinal tract of the patient so that gastro-intestinal content pass through the open portion.

The attractor assembly may be made in part of a ferromagnetic material and may include at least one permanent magnet. The magnet assembly may be with the first assembly and include one elongated permanent magnet or a plurality of separate permanent magnets extending at least partially around the portion of the gastrointestinal tract. The first assembly may define a gap with the first assembly positioned around the patient's gastrointestinal tract, with the gap closed to retain the first assembly against an outer surface of the gastrointestinal tract. The first and/or second assembly may be made at least in part from a bio-absorbable material. The technique may be used to treat obesity, a metabolic disease, gastric reflux, and/or hiatal hernia.

A method of stimulating baro receptors at the cardiac portion of the stomach, and an intraluminal device, according to an aspect of the invention, includes fixing an intraluminal device to the cardiac portion of the stomach with a magnetic fastener. The intraluminal device includes a wall defining a baro receptor stimulating surface,. The baro receptor stimulating surface and the cardiac portion of the stomach are conformable with each other. The magnetic fastener having a magnet assembly generating magnetic flux and an attractor assembly being attracted to the magnet assembly in response to the magnetic flux. A first assembly, having either the magnet assembly or the attractor assembly, is fixed against the inner surface or an outer surface of the stomach at the cardiac portion of the stomach. A second assembly, having the wall and the other of the magnet assembly or attractor assembly, is deployed to the portion of the inner surface the stomach. Magnetic attraction between the magnet assembly and attractor assembly applies stress, with the baro receptor stimulating surface, to stimulate baro receptors at the cardiac portion of the stomach.

The first assembly may be deployed to the abdominal cavity and positioned against the outer surface of the stomach or deployed to the interior of the stomach and fixed to the cardiac portion of the stomach. The second assembly may have an open portion of said baro receptor stimulating surface. The first assembly may magnetically generally align the open portion with the GE junction of the patient so that food passes through the open portion.

The magnet assembly may be with the second assembly and include one or more permanent magnets in the stomach generating magnetic flux through the stomach wall. The amount of stress applied with the baro receptor stimulating surface to the cardiac portion of the stomach may be selected by replacing the second assembly with a second assembly producing a different amount of magnetic flux than the removed magnet assembly. The second assembly may be replaced with a second assembly having a different shape or contact area of the baro receptor stimulating surface, or different stiffness of the wall. Alternatively, the amount of stress applied with the baro receptor stimulating surface to the cardiac portion of the stomach may be selected by configuring the first and/or second assemblies to select a particular separation distance of the permanent magnet from the attractor assembly.

The stress applied to the cardiac portion of the stomach with said intraluminal device may be made selectable with a controller. The controller may be with the second assembly and adapted to adjust stress applied by the surface to the cardiac portion of the stomach by modifying shape, area, or amount of contact between the surface and the cardiac portion of the stomach. The controller may be adapted to adjust stress applied by the baro receptor stimulating surface to the cardiac portion of the stomach including adjusting amount of current to at least one electro-magnet. The magnet assembly may include at least one electro-magnet combined with at least one permanent magnet, with the controller adapted to adjust magnetic flux produced by the at least one permanent magnet. The magnet assembly may include a plurality of electro-magnets distributed about the wall of the stomach, each paired with a permanent magnet and adapted to adjust amount of magnetic flux produced by its paired permanent magnet. The controller may be adapted to adjust stress applied by the baro receptor stimulating surface to the cardiac portion of the stomach by (i) supplying current selectively to the electro-magnets to choose which ones of said permanent magnets are engaged with or disengaged from the attractor assembly, or (ii) adjusting amount of attraction between said permanent magnets and the attractor assembly by adjusting amount of current supplied to the electromagnets.

The invention can be embodied in endoscopic and/or laparoscopic techniques. The intraluminal device can be easily replaced with an intraluminal device adapted to generating more or less or a variable amount of stress to respond to the needs of the patient. Pressure, or stress, applied to the stomach wall may engage barro or stretch receptors in the stomach wall.

Peristalsis in the stomach wall may also create a sense of fullness by causing relative movement between the stomach wall and the wall of the intraluminal device thus activating the barro receptors. Activating of the barro receptors may cause neurohormonal change in the patient and trigger brain activity to affect satiety.

While the invention is illustrated with an intraluminal device that is fixed to the wall of the cardiac portion of the stomach in order to stimulate baroreceptors, it may have applications in fixing other devices to other portions of the gastrointestinal tract.

These and other objects, advantages, purposes and features of this invention will become apparent upon review of the following specification in conjunction with the drawings.

### BRIEF DESCRIPTION OF THE DRAWINGS

FIG. 1 is a perspective view of an intraluminal device deployed to a patient according to an embodiment of the invention;

FIG. 2 is a sectional view taken along the lines II-II in FIG. 1;

FIG. 3 is a sectional view taken along the lines III-III in FIG. 1;

FIG. 4 is a top plan view of an alternative embodiment of an abdominal member;

FIG. 5 is the same view as FIG. 4 of another alternative embodiment of an abdominal member;

FIG. 6 is the same view as FIG. 4 of yet another alternative embodiment of an abdominal member;

FIG. 7A is a block diagram of a magnetic fastener;

FIG. 7B is a block diagram of an alternative embodiment of a magnetic fastener;

FIG. 7C is a block diagram of another alternative embodiment of a magnetic fastener;

FIG. 8A-8E are perspective views of alternative embodiments of an intraluminal device;

FIG. 9 is a block diagram of a deployment system that is adapted to deploy an intraluminal device to the cardiac region of the stomach;

FIG. 10 is the same view as FIG. 1 of an alternative embodiment of the invention;

FIG. 11 is an enlarged perspective view of the intraluminal device in FIG. 10 illustrating individual patches in various orientations;

FIG. 12 is a sectional view of an intraluminal device deployed to a patient according to another embodiment of the invention;

FIG. 13 is a top plan view of an intraluminal device according to another embodiment of the invention; and

FIG. 14. is a flow diagram of a method according to an embodiment of the invention.

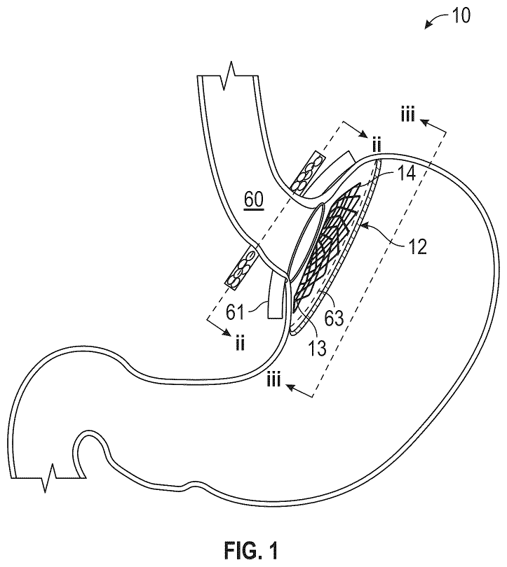

### DESCRIPTION OF THE PREFERRED EMBODIMENTS

The present invention will now be described with reference to the accompanying figures, wherein the numbered elements in the following written description correspond to like-numbered elements in the figures. A method 10 is provided for deploying an intraluminal device 12 to the cardiac region of the stomach. The intraluminal device 12 includes a wall 13 that defines a surface 14 that generally conforms to the cardiac portion of the stomach. An open portion, such as a through-hole 16, is defined in wall 13 and is alienable with the gastroesophageal (GE) junction of the patient. While open portion 16 is a generally central, in wall 12 it could alternatively be a notch or other shape depending on the configuration of the intraluminal device, or could be a multiple member intraluminal device whose combination leaves an open portion between the members. Wall 13 and the cardiac portion of the stomach are conformable to each other in order to stimulate baroreceptors in the wall of the cardiac portion of the stomach. This may be accomplished by fixing wall 13 to the stomach wall bringing the device wall and the stomach wall into physical contact in order to apply stress, or pressure, to the wall of the stomach. Alternatively, device wall 13 and the stomach wall may be more loosely joined so that the peristalsis of the stomach will bring the stomach wall into engagement will the device wall to stimulate baroreceptors. Alternatively, or additionally, the intraluminal device may include an internal and/or external control that is adapted to vary the stress as will be discussed below. Detail operation of intraluminal device 12 is described in commonly assigned U.S. Pat. No. 7,846,174 and Patent Application Publication US 2016/0151233, the disclosures of which are hereby incorporated herein by reference in their entirely.

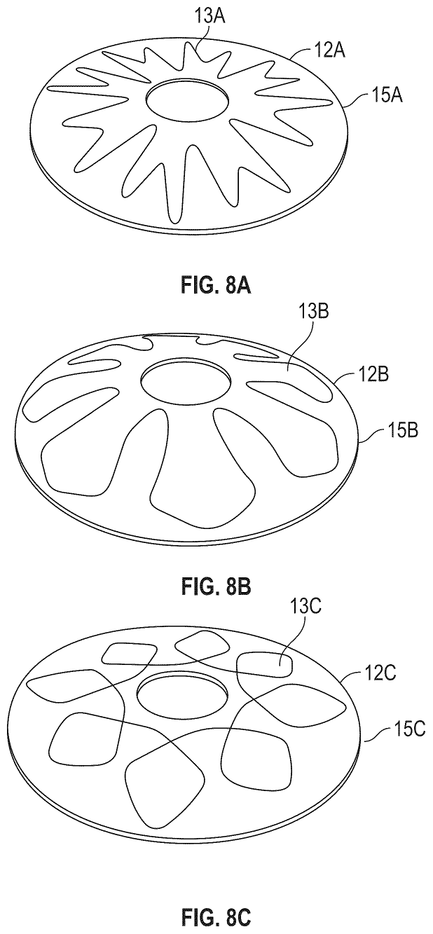

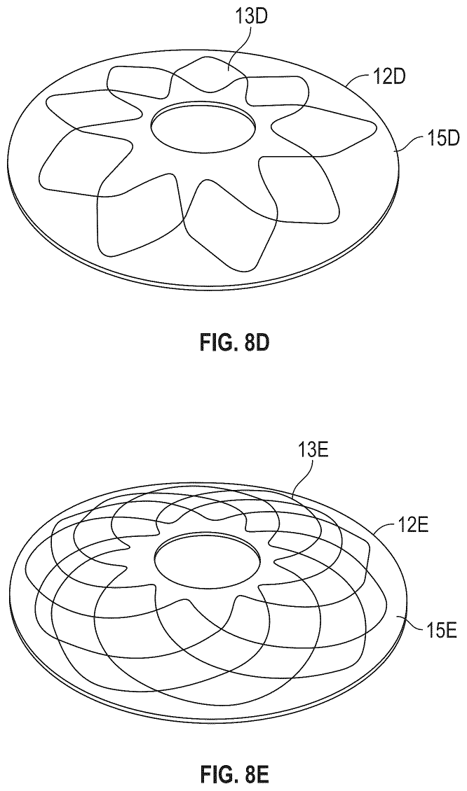

Amount of pressure applied by wall 13 may be varied by varying the stiffness of intraluminal device 12. This may be accomplished by varying the amount of silicone in flexible membrane 15, which may be different in different portions of the device. Alternatively, the material and/or structure of the structural element 13 of the of the intraluminal device may be varied. Various configurations of wall 12A-12E are illustrated in FIG. 8A though 8E. Each wall configuration is constructed of a structural element 13A-13E and a flexible membrane 15A-15E between loops of the structural element. Structural element 13A-13E can be made from any suitable material that can provide rigidity but be flexible, such as a metal such as nitinol, or a polymeric material or carbon filament of the type known in the art. Structure element 13A-13E various shapes provide enough rigidity to wall 12A-12E in order to apply suitable stress to the cardiac portion of the stomach without concentrating stress at particular locations. Yet, wall 12A-12E is flexible enough to be able to be compressed to an endoscopic deployment device, of the type known in the art. Flexible membrane 15A-15E can be made from a biocompatible material such as silicone, or the like, and of a suitable thickness to provide rigidity but be flexibility.

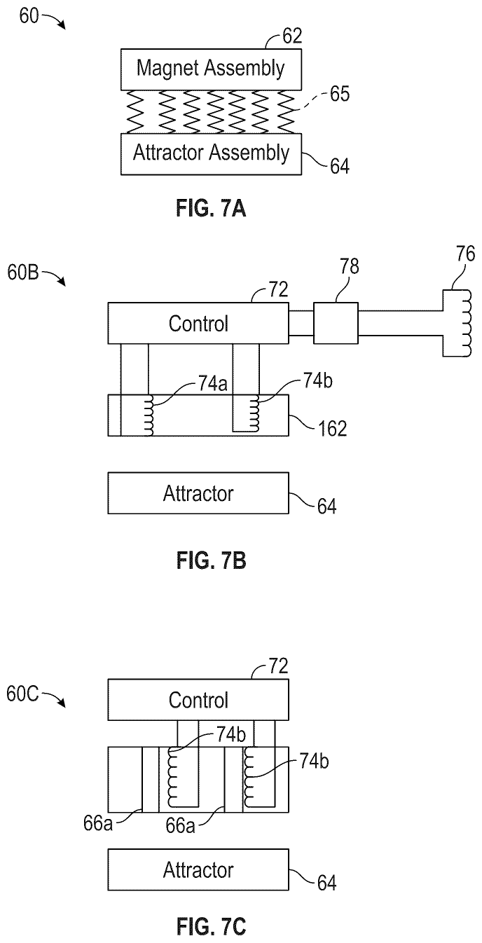

In order to stimulate baroreceptors, intraluminal device 12 is fixed to a portion of the inner wall of the gastrointestinal tract, which in the illustrated embodiment is the cardiac portion of the stomach, with one or more magnetic fasteners 60. Magnetic fastener 60 includes a magnet assembly 62 and an attractor assembly 64. As best illustrated in FIG. 7A, magnet assembly 62 generates magnetic flux 65 and attractor assembly 64 responds to the magnetic flux by being attracted to magnet assembly 62. Flux 65 is generated by magnet assembly 62 by either one or more permanent magnets 66, one or more electro-magnets 74 or a combination of both. Attractor assembly 64 is Ferro-magnetic and may or may not be permanently magnetized. If Attractor assembly 64 defines one or more permanent magnets, magnetic poles of the magnet assembly and attractor assembly should be taken into account in device design as is understood in the art. Unlike magnetic poles facing each other will attract. Like poles facing each other will repel. Since stomach wall tissue is being compressed between the magnet assembly and the attractor assembly, care should be taken to minimize widespread necrosis of tissue at the deployment cite. This can be accomplished, for example by supplying more small diameter magnetic fasteners rather than fewer large ones. Thus only a small area of the stomach wall is captured between each magnet and attractor. While this may result in necrosis in the immediate location of the magnetic fastener, that area will form scar tissue and the surrounding area will be well supplied with blood vessels. So the overall tissue of the stomach wall will remain healthy. Also, channels may be defined in tissue engaging surfaces of the abdominal assembly and/or stomach assembly at the location of blood vessels in order to avoid pinching off the vessels.

Intraluminal device 12 includes a first assembly 61, which in the illustrated embodiment, is an abdominal assembly that is adapted to be deployed to the abdominal cavity of the patient in close proximity to or contact with the outer wall of the top portion of the stomach. First assembly 61 may include the magnet assembly 62 or the attractor assembly 64, depending upon the application. Intraluminal device 12 includes a second assembly 63 which, in the illustrated embodiment, is a stomach assembly that is adapted to be deployed to the stomach of the patient in contact with the cardiac portion of the stomach opposite from the abdominal assembly. While they could be deployed separately to the stomach, in most embodiments the stomach assembly 63 includes the intraluminal device having wall 12 and the magnet assembly 62 or attractor assembly 64. While components making up stomach assembly 63 could be deployed separately and assembled in situ, in the illustrated embodiments, stomach assembly 63 is assembled before deployment and deployed as a unit.

Whether magnet assembly 62 or attractor assembly 64 is used with stomach assembly 63 will depend upon which one is used with the abdominal assembly 61. As will be discussed in more detail below, in addition to attracting the attractor assembly toward the stomach wall, magnetic flux 65 will tend to align the attractor assembly with the magnet assembly against the stomach wall in the position directly opposite each other. The location of the abdominal assembly generally around the upper stomach, will ensure a through-path for food from the GE junction and through-opening 16 to the stomach for further digestion because the magnetic flux will tend to align the magnet assembly with the attractor assembly.

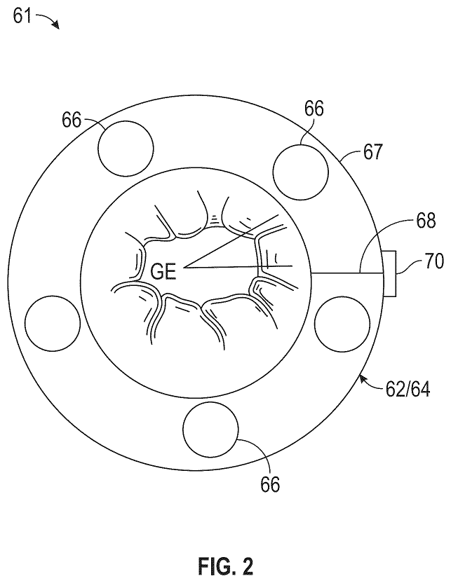

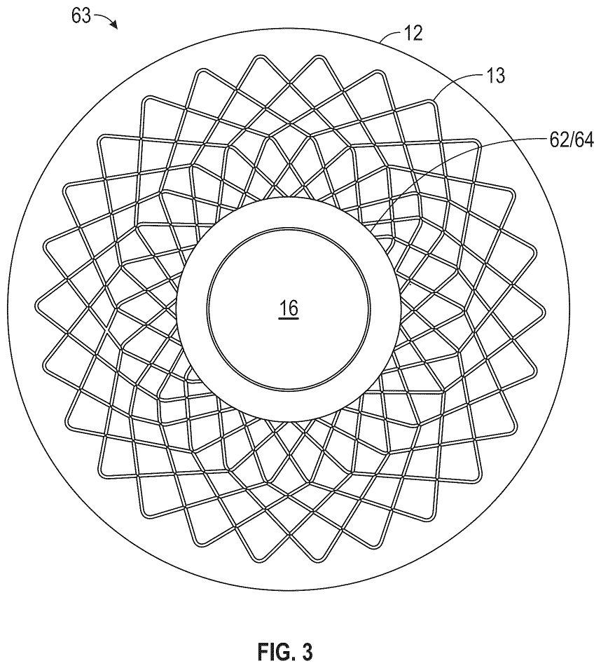

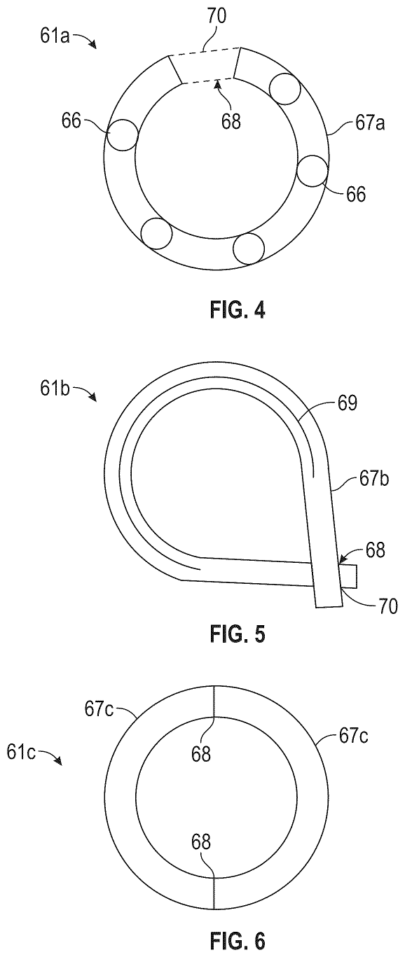

Intraluminal device 12 includes abdominal assembly 61 being deployed to the abdominal cavity, such as using laparoscopic instruments. Abdominal assembly 61 is configured to be positioned against the stomach wall opposite the cardiac portion of the stomach and retained in that position, as illustrated in FIG. 2. As illustrated in FIG. 4, this may accomplished by forming an abdominal assembly 61a in a horse-shoe shape defining a gap 68 that is sized to allow the abdominal assembly 61a to be passed around the upper stomach. Once the abdominal assembly is positioned around the upper stomach, a closure 70 is applied in order to retain the abdominal assembly in this position. This configuration allows abdominal assembly 61a to be deployed around the stomach using the pars flacida procedure, which is known in the art.

Alternative embodiments of an abdominal assembly are illustrated in FIGS. 5 and 6. In contrast to abdominal assembly 61a which includes a plurality of permanent magnets spaced about a substrate, abdominal assembly 61b is made up of a linear, or strip, magnet 69 mounted to a substrate 67. Both configurations distribute a loosely defined cylinder of magnetic flux that is generally centered on the geographic center of substrate 67, 67a, which guides the stomach assembly into position with open portion 16 sufficiently aligned with the GE junction. Substrate 67a has overlapping arms 71 which define a gap 68a. Substrate 67a is defined in part by material having shape memory so that arms 71 may be spread apart in order to deploy the abdominal assembly around the upper stomach. Once so positioned, arms 71 return to their original shape position with the arms overlapping in order to retain its position around the upper stomach. An abdominal assembly 61c is made up of separate substrate pieces 67c, each bearing permanent magnet(s). Substrate pieces 67c are semicircular in shape defining gap pairs 68 that facilitate pieces 67c to encircle the upper stomach before they are joined. One or more closures 70, such as clasps or pivots may be used to combine substrate pieces 67c. Other modifications will be apparent to the skilled artisan, such as configuring the substrate pieces 67C using permanent magnets having opposite poles so that they are attracted to each other around the upper stomach. In such embodiment, closures 70 are optional.

In an alternative embodiment, the abdominal assembly is deployed to the abdomen via the stomach using the techniques disclosed in commonly assigned International Application Serial No. ______ filed even date with the present application and entitled METHOD OF FIXATION OF INTRALUMINAL DEVICE, INTRALUMINAL FIXATION SYSTEM AND INTRALUMINAL FASTENER, the disclosure of which is hereby incorporated by reference in its entirety. In this embodiment, the abdominal member is formed by a magnetized alloy capable of memory shape. Such magnetized alloy can be deployed to the abdomen through the stomach wall from inside the stomach where its shape memory can be formed to surround the upper stomach. For example, the shape memory may form a flower pedal, circle, or the like. Once deployed in this fashion, the magnetized alloy may be retrieved from the stomach by leaving a portion extending into the stomach. Alternatively, the deployment opening can be closed when retrieval in this fashion is not desired. Such magnetized alloy capable of memory shape can be provided, for example, by supplying a nickel layer to nitinol which is then magnetized. With the magnetized alloy around the upper stomach, an attractor assembly in the stomach will be attracted to the cardiac portion of the stomach. Alternatively, the memory shape may be made from a ferro-magnetic material to be attracted by magnetic flux generated by the stomach assembly.

In the illustrated embodiment, stomach assembly 63 is deployed endoscopically to the stomach. Once unfurled in the stomach, magnetic attraction between magnet assembly 62 and attractor assembly 64 attracts surface 14 of wall 12 against the inner wall of the gastro-intestinal tract and thereby applies stress to the baroreceptors of the cardiac portion of the stomach. Moreover, the magnetic attraction between magnet assembly 62 and attractor assembly 64 causes the stomach assembly to generally align with the abdominal assembly on opposite sides of the stomach wall as previously described. Since the abdominal assembly is retained in a position around the upper stomach, magnetic attraction between these two assemblies will align (or at least cause sufficient overlap), the open portion 16 of the intraluminal device and the GE junction of the patient so that food traversing the esophagus passes to the stomach and not be diverted such as to between surface 14 and the stomach wall.

In an embodiment, an intraluminal device is adapted to select, or adjust, an amount of stress applied to inner wall of the gastro-intestinal tract, such as the cardiac portion of the stomach. In such embodiment, stomach assembly 43 includes magnet assembly 62, with the abdominal assembly including attractor assembly 64. Adjustability may be accomplished, according to this embodiment, by removing stomach assembly 43 and replacing it with a difference stomach assembly having a magnet assembly 62 adapted to generate a stronger or weaker magnetic flux 65. This is a relatively simple procedure since the stomach assembly is held in place magnetically and can be removed and replaced without the need for accessing the abdomen or severing sutures or the like. Since the replacement is in the stomach, it can be performed endoscopically using essentially the same procedure used to initially deploy the stomach assembly. Such replacement may be used, for example, to increase stress on the cardiac portion of the stomach to increase weight loss, or to decrease stress after the patient has experienced substantial weight loss, or the like.

An alternative embodiment 60B of a magnetic fastener is shown in FIG. 7B. Magnetic fastener 60B has a magnet assembly 162 with one or more electro-magnets 74a, each electrically energized by an electrical control 72 to produce magnetic flux 65. In this manner, control 72 can adjust flux 65 by controlling amount of electrical current and/or which electro-magnets receive current. Electrical energy can be supplied inductively to control 72 via a sub-cutaneous induction coil 76 and a storage media 78, such as a re-chargeable battery or super-capacitor.

Another alternative embodiment 60° C. of a magnetic fastener is shown in FIG. 7C. In magnetic fastener 60C, each electro-magnet 74b is paired with a permanent magnet 66a in a magnet assembly 262. Such combination is known in the art as an electro-permanent magnet and uses a combination of a permanent magnet and an electro-magnetic to create the magnetic flux. Once the magnetic flux is established, it can be maintained by the permanent magnet without continuous power supply. This allows control 72 to supply current to electro-magnets 74b in order to cause the paired permanent magnet 66a to attract the corresponding portion of attractor assembly 64 to increase stress applied by intraluminal device 10 to the cardiac portion of the stomach. In a similar fashion, a reverse current supplied by control 72 to electro-magnet 74b can reduce attraction with the corresponding portion of attractor assembly 64. Magnetic fastener 260 is more energy efficient because the permanent magnet can generate the majority of the magnetic flux and the electro-magnet used to make changes to the flux generated by the permanent magnet. Magnetic fasteners 160, 260 may be applied to the abdominal assembly. This is especially useful since the abdominal assembly can remain in the abdominal cavity even as stomach assembly is removed and replaced.

Alternatively, a magnetic fastener may have control placed in the stomach with the stomach assembly along with the magnet assembly in order to control amount of attraction between the magnet assembly and attractor assembly. Alternatively, a separate control may be combined with the stomach assembly in order to adjust amount of stress applied to the cardiac portion of the stomach. Examples may include controllable bladders between the device surface and cardiac portion of the stomach, or controllable filaments that are adapted to adjust amount of surface area in contact with the stomach, using techniques disclosed in commonly assigned International Application Publication No. WO 2020/183399, the disclosure of which is hereby incorporated herein by reference in its entirety.

Visualizing of the intraluminal device in the stomach, such as with an endoscope may optionally be useful to ensure proper placement of the stomach assembly. Also, the deployment tool that deploys the intraluminal device in the stomach can be used to further ensure alignment between the open portion of the intraluminal device and the GE junction.

Other features of the Intraluminal Device and deployment technique disclosed in commonly assigned International Application Publication No. WO 2023/166472, the disclosure of which is hereby incorporated herein by reference in its entirety, may further be useful with the embodiments disclosed herein. For example, the intraluminal device may be deployed over the endoscope as a deployment tool. Also, the various techniques may be used to control stress applied to the cardiac portion of the stomach. Examples controllable bladders may be provided between the device surface and cardiac portion of the stomach, or controllable filaments adjusted by a control to titrate amount of surface area of the intraluminal device in contact with the stomach.

FIGS. 8A through 8E show various configurations of intraluminal devices 12A-12E with configurations of structural members or elements 13A-13E. Other configurations are possible. For example, segments of the structural members may be denser proximal the open portion of the intraluminal device than distal thereto.

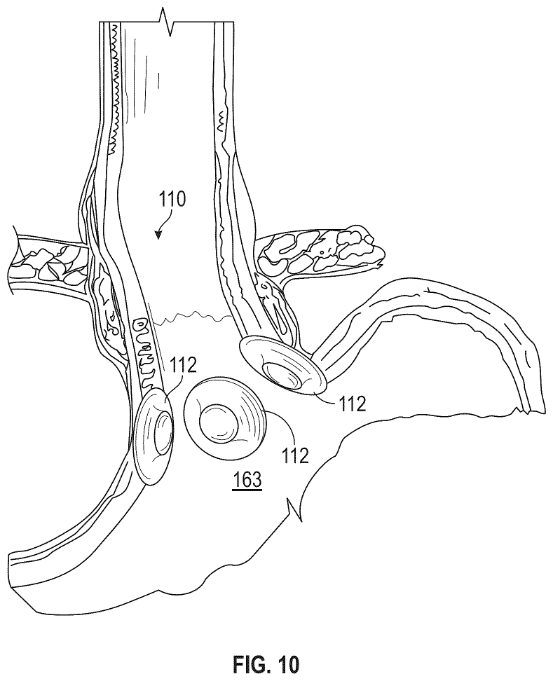

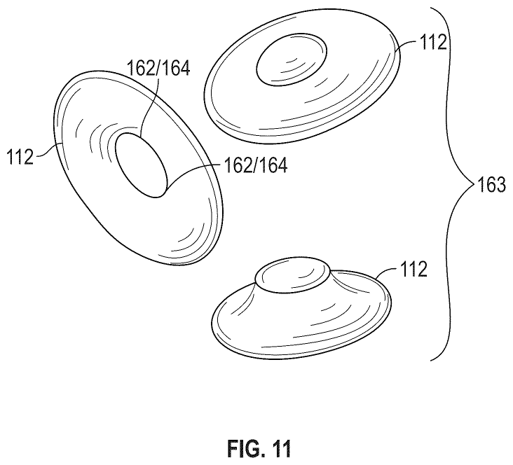

Alternatively, in other embodiments the wall defining the surface of the stomach assembly may be no larger than required to engage with the magnetic field generated by a magnetic assembly or attractor assembly in the abdomen, such as a narrow ring shape or the like. In another alternative embodiment a method 110 includes a stomach assembly 163 that is defined by a plurality of individual patches 112 each having a separate magnet assembly 162 or an attractor assembly 164 (FIGS. 10 and 11). The individual patches 112 are deployed to the stomach where whey will tend to align with the portion of the magnetic fastener in the abdomen. Since the portion of the magnetic fastener in the abdomen is distributed around the upper stomach, the individual patches will tend to distribute themselves around the GE junction. Such configuration may provide a particular level of stimulus of the baroreceptors in the cardiac portion of the stomach which may be desired for a particular patient characteristic and the level of stimulus may be varied by changing the number of individual patches deployed to the stomach. Intraluminal device 12, 112, 212 may have various uses. The intraluminal device may be used as a bariatric device. The intraluminal device may be used to treat a metabolic disease. The intraluminal device tends to return the upper region of the stomach to its anatomically correct configuration when deployed. This tends to resist upward movement to the stomach wall and may be used to treat gastric reflux disease or hiatal hernia.

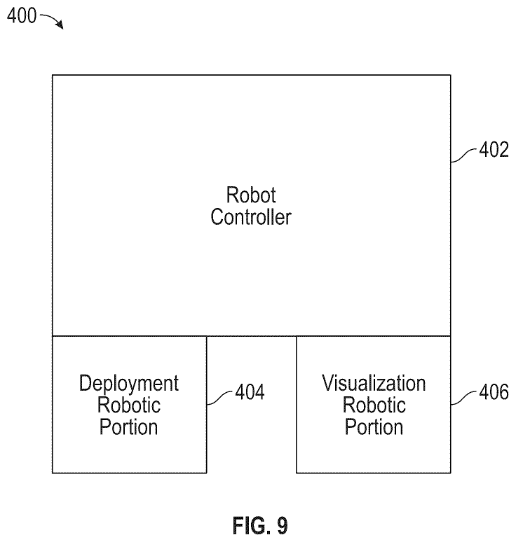

Illustrated in FIG. 9 is a surgical robotic system 400 that is useful in carrying out the invention, although humans can alternatively carry out embodiments of the invention. Robotic system 400 has a device deployment robotic portion end-of-arm tool 404 that deploys the stomach assembly to the stomach and removes the stomach assembly after use, or to interchange stomach assemblies to titrate the amount of stress being applied by intraluminal device 12. Robotic system 400 further has a visualization robotic portion 406 that visualizes the interior of the stomach while the intraluminal device is being fastened. A robotic control 402 adjusts the relative movement between tools 404 and 406 to ensure that the intraluminal device is properly engaged by the fasteners and that the anatomy of the patient is not compromised by the deployment process.

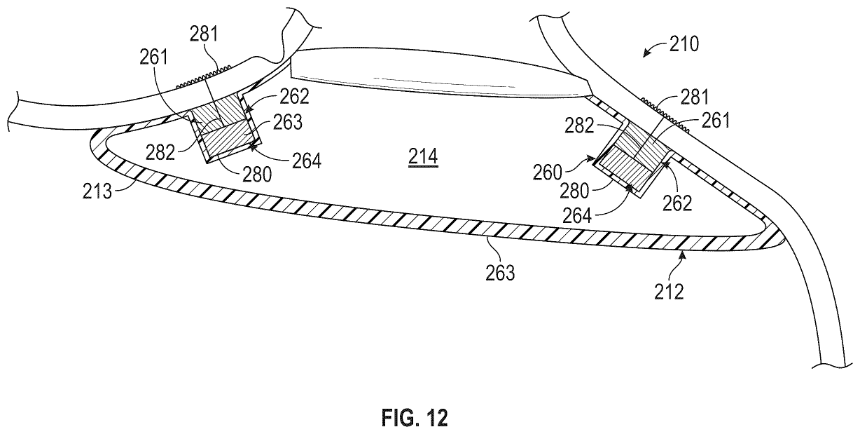

In yet another alternative embodiment, method 210 of fixing intraluminal device 212 according to an embodiment of the invention, both a magnet assembly 262 and attractor assembly 264 are deployed to the stomach (FIG. 12). Method 210 includes an intraluminal device 212 having a wall 213 defining a surface 214 that is adapted to conform generally to the contour of the cardiac portion of the stomach. Intraluminal device 212 is fixed to a portion of the inner wall of the gastrointestinal tract with one or more magnetic fasteners 260. Magnetic fastener 260 includes a magnet assembly 262 and an attractor assembly 264. Intraluminal device 212 includes a first assembly 261 that is positioned within the stomach and fixed to the stomach wall. First assembly 261 may include the magnet assembly 262 or the attractor assembly 264, depending upon the application. Intraluminal device 212 includes a second assembly 263 which is deployed to the stomach of the patient in contact with the cardiac portion of the stomach opposite from the abdominal assembly.

In the illustrated embodiment, first assembly 261, which includes either the magnet assembly 262 or the attractor assembly 264, is fixed to the interior surface of the stomach wall with a filament 282, such as a non-absorbable suture, passing through the stomach wall to a scared area formed at the outer surface of the outer wall of the stomach. Such a scared area, which resists the filament from eroding through the stomach wall, may be formed by a pledget 281 made from a bio-absorbable material as disclosed in International Publication No., WO 2023/166472, the disclosure of which is hereby incorporated herein by reference. Filament 282 could be passed with an endoscopic needle through the first assembly and through the stomach wall where it is grasped by a laparoscopic instrument. Other fastening techniques are possible, such as disclosed in the commonly assigned International Application filed even date with the present application and entitled METHOD OF FIXATION OF INTRALUMINAL DEVICE, INTRALUMINAL FIXATION SYSTEM AND INTRALUMINAL FASTENER.

Second assembly 263 includes one or more pocket 280 formed in wall 213 which positions the other one of the either the magnet assembly 262 or the attractor assembly 264 recessed with respect to the baro receptor stimulating surface 214. This Provides sufficient room to receive other of the magnet assembly or attractor assembly. Thus, with magnet assembly 262 and attractor assembly 264 defining opposite magnetic poles, their magnetic attraction will attract intraluminal device 212 toward the cardiac portion of the stomach with baro receptor stimulating surface 214 stimulating baro receptors at the wall of the cardiac portions of the stomach.

Method 210 has several advantages. By positioning both the first assembly and second assembly in the stomach, the magnetic attraction between magnet assembly 262 and attractor assembly 264 does not need to pass through the stomach wall. Thus the stomach wall does not get compressed between the magnet and attractor assembly so provisions are not required to prevent necrosis of stomach wall tissue. Also, because the first assembly is positioned in the stomach and may be endoscopically deployed, deployment may be simpler than doing so laparoscopically. If the first assembly is affixed fully endoscopically, then no access to the abdominal cavity will be required.

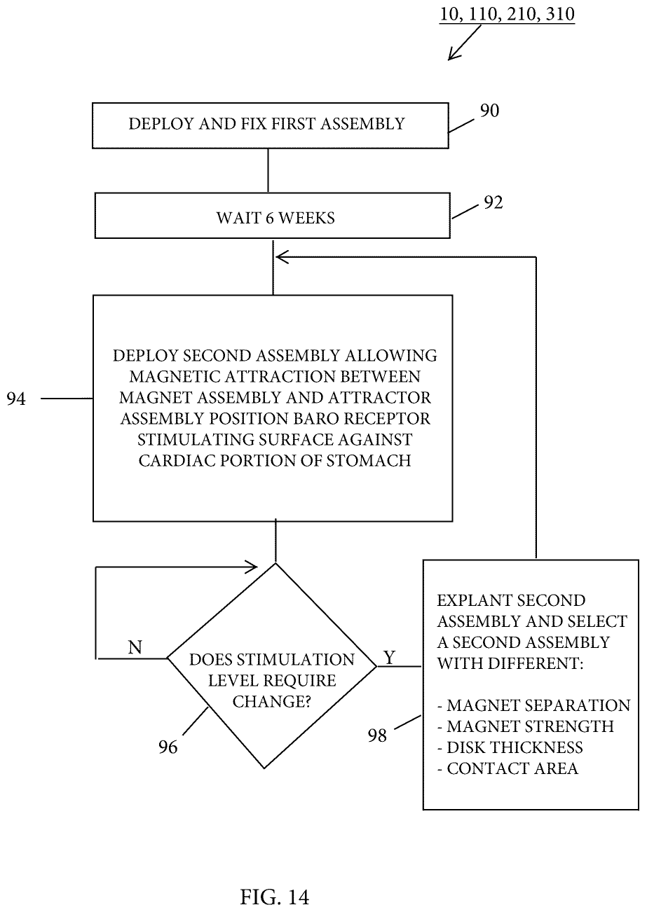

Operation of method 10, 110, 210, 310 is illustrated in FIG. 14. The first assembly is deployed at 90. In method 10, the first assembly is deployed laparoscopically to the outer wall of the stomach. In method 210, the first assembly is deployed endoscopically to the inner wall of the cardiac portion of the stomach. In method 110 the first assembly could be deployed to the outer wall or the inner wall of the stomach. Where an absorbable pledget is used, a wait of 6 weeks is incorporated at 92. This allows time for formation of scar tissue to help strengthen the fixation. The second assembly is then deployed to the stomach at 94. As the attractor assembly is brought into influence of the flux generated by the magnet assembly, the magnetic attraction positions the baro receptor stimulation surface 14, 114, 214, 314 against the cardiac portion of the stomach to stimulate the baro receptors.

It is then determined at 96 whether the level of stimulation of the baro receptors is no longer appropriate. This may be a result, for example, of the patient having lost sufficient body mass to require less stimulation, or the patient loosing body mass too slowly or too quickly. If it is determined at 96 that the level of stimulation requires changing, the second assembly is explanted at 98 and replaced with a different second assembly with a different stimulation level.

Change in stimulation level may be accomplished at 98 by varying one or more operating parameters of the intraluminal device. Changes may be accomplished, for example, through variation of magnetic attraction strength, variation of baro receptor stimulation contact area or contact force, variation in stiffness of the disk, variation of thickness and/or distribution of material in the disk, weave or material of the structure wire, and the like.

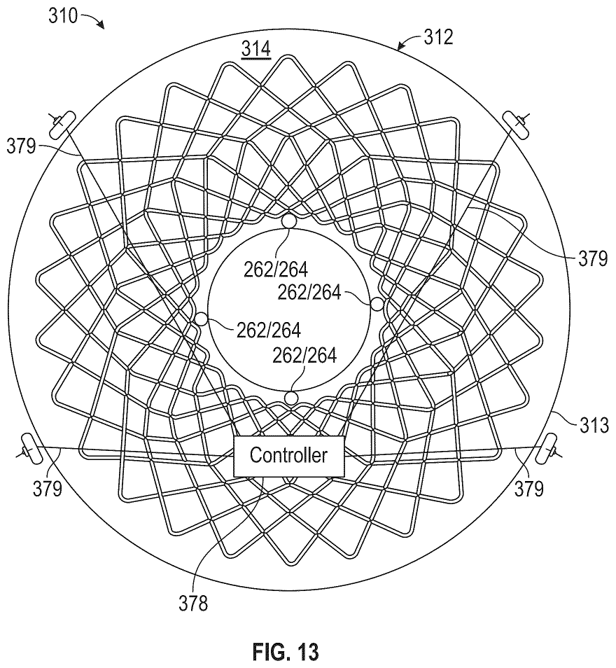

A method 310 utilizes an intraluminal device 312 having a baro receptor stimulating surface 314 and a controller 378 (FIG. 13). Controller 378 is connected with wall 313 by a plurality of filaments 379 which can be extended or retracted by controller 378. As filaments 379 are shortened, a greater portion of wall 313 is pulled away from the cardiac portion of the stomach, thus reducing contact between the baro receptor stimulating surface 314 and the cardiac portion of the stomach. Conversely, as filaments 379 are allowed to lengthen, wall 313 will return to a natural shape with more of baro receptor stimulating surface 314. Thus controller 378 is able to control amount of surface contact between intraluminal device 312 and the cardiac portion of the stomach.

Changes and modifications in the specifically described embodiments can be carried out without departing from the principles of the present invention which is intended to be limited only by the scope of the appended claims, as interpreted according to the principles of patent law including the doctrine of equivalents.

1.-4. (canceled)

5. A method of stimulating baro receptors at the cardiac portion of the stomach, comprising:
fixing an intraluminal device to the cardiac portion of the stomach with a magnetic fastener, said intraluminal device comprising a wall defining a baro receptor stimulating surface, said baro receptor stimulating surface and the cardiac portion of the stomach being conformable with each other, said method comprising:
said magnetic fastener having a magnet assembly and an attractor assembly, said magnet assembly generating magnetic flux and said attractor assembly being attracted to the magnet assembly in response to the magnetic flux;
deploying a first assembly comprising said magnet assembly or said attractor assembly against the inner surface or an outer surface of the stomach at the cardiac portion of the stomach;
deploying a second assembly having a wall and the other of said one chosen from said magnet assembly and said attractor assembly to the portion of the inner surface the stomach; and
wherein magnetic attraction between said magnet assembly and said attractor assembly applies stress with the baro receptor stimulating surface to stimulate baro receptors at the cardiac portion of the stomach; and
adjusting level of stimulation of the baro-receptors.

6. The method as claimed in claim 5 wherein said first assembly is deployed to the abdominal cavity and positioned against the outer surface of the stomach or is deployed to the interior of the stomach and fixed to the cardiac portion of the stomach.

7. The method as claimed in claim 5 wherein said second assembly has an open portion of said baro receptor stimulating surface and wherein the first assembly magnetically generally aligns the open portion with the GE junction of the patient so that food passes through the open portion.

8. The method as claimed in claim 7 including positioning said first assembly around the upper portion of the stomach.

9. The method as claimed in claim 5 wherein magnet assembly is with said second assembly and comprises at least one permanent magnet in the stomach generating magnetic flux.

10. The method as claimed in claim 9 including selecting amount of stress applied with the baro receptor stimulating surface to the cardiac portion of the stomach by replacing said second assembly with a second assembly producing a different stimulation level.

11. The method as claimed in claim 9 including selecting amount of stress applied with the baro receptor stimulating surface to the cardiac portion of the stomach by replacing said second assembly with a second assembly having variation of at least one selected from (i) magnetic attraction strength, (ii) baro receptor stimulation contact area, (iii) baro receptor stimulation contact force, (iv) stiffness of the wall, (v) variation of thickness and/or distribution of material in the wall, (vi) weave of a structure wire, and (vii) material of the structure wire.

12. The method as claimed in claim 9 including selecting amount of stress applied with the baro receptor stimulating surface to the cardiac portion of the stomach by configuring at least one chosen from said first and second assemblies to selecting a separation of the permanent magnet from the attractor assembly.

13. The method as claimed in claim 5 wherein said attractor assembly comprises a ferromagnetic material.

14. The method as claimed in claim 5 wherein said attractor assembly includes at least one permanent magnet.

15. The method as claimed in claim 5 wherein said magnet assembly is with said first assembly and comprises at least one elongated permanent magnet extending at least partially around the portion of the gastrointestinal tract.

16. The method as claimed in claim 5 wherein said magnet assembly is with said first assembly and comprises a plurality of separate permanent magnets mounted to a substrate extending at least partially around the portion of the gastrointestinal tract.

17. The method as claimed in claim 16 wherein said first assembly defining a gap and including positioning the first assembly around the patient's gastrointestinal tract and selectively closing said gap to retain the first assembly against an outer surface of the gastrointestinal tract.

18. The method as claimed in claim 5 including adjusting stress applied to the cardiac portion of the stomach with said intraluminal device with a controller.

19. The method as claimed in claim 18 wherein said controller is with the second assembly and is adapted to adjust stress applied by the surface to the cardiac portion of the stomach including modifying shape, area, or amount of contact between the surface and the cardiac portion of the stomach.

20. A method of stimulating baro receptors at the cardiac portion of the stomach, comprising:
fixing an intraluminal device to the cardiac portion of the stomach with a magnetic fastener, said intraluminal device comprising a wall defining a baro receptor stimulating surface, said baro receptor stimulating surface and the cardiac portion of the stomach being conformable with each other, said method comprising:
said magnetic fastener having a magnet assembly and an attractor assembly, said magnet assembly generating magnetic flux and said attractor assembly being attracted to the magnet assembly in response to the magnetic flux;
deploying a first assembly comprising said magnet assembly or said attractor assembly against the inner surface or an outer surface of the stomach at the cardiac portion of the stomach;
deploying a second assembly having a wall and the other of said one chosen from said magnet assembly and said attractor assembly to the portion of the inner surface the stomach; and
wherein magnetic attraction between said magnet assembly and said attractor assembly applies stress with the baro receptor stimulating surface to stimulate baro receptors at the cardiac portion of the stomach;
adjusting level of stimulation of the baro-receptors with a controller.
wherein said controller is adapted to adjust level of stimulation of the baro-receptors by controlling stress applied by the baro receptor stimulating surface to the cardiac portion of the stomach including adjusting amount of current to at least one electro-magnet.

21. The method as claimed in claim 20. wherein said magnet assembly includes at least one electro-magnet combined with at least one permanent magnet, and said controller is adapted to adjust magnetic flux produced by said at least one permanent magnet.

22. The method as claimed in claim 21 said magnet assembly including a plurality of electro-magnets distributed about the wall of the stomach, each paired with a permanent magnet and adapted to adjust amount of magnetic flux produced by its paired permanent magnet, wherein said controller is adapted to adjust stress applied by the baro receptor stimulating surface to the cardiac portion of the stomach by (i) supplying current selectively to the electro-magnets to choose which ones of said permanent magnets are engaged with or disengaged from the attractor assembly, or (ii) adjusting amount of attraction between said permanent magnets and the attractor assembly by adjusting amount of current supplied to the electromagnets.

23. The method as claimed in claim 5 wherein at least one selected from said first assembly and said second assembly are made at least in part from a bio-absorbable material.

24. The method as claimed in claim 5 used to treat at least one chosen from obesity, a metabolic disease, gastric reflux, and hiatal hernia.

25. (canceled)

26. An intraluminal device that is adapted to apply stress to the cardiac portion of the stomach, comprising:
a magnetic fastener;
a wall defining a baro receptor stimulating surface, said baro receptor stimulating surface and the cardiac portion of the stomach conformable with each other;
said magnetic fastener having a magnet assembly and an attractor assembly, said magnet assembly generating magnetic flux and said attractor assembly being attracted to the magnet assembly in response to the magnetic flux;
a first assembly comprising said magnet assembly or said attractor assembly, said first assembly configured to be fixed against the inner surface or an outer surface of the stomach at the cardiac portion of the stomach;
a second assembly having said wall and the other of said one chosen from said magnet assembly and said attractor assembly, said second assembly adapted to be deployed to the stomach;
wherein the magnetic attraction between said magnet assembly and said attractor assembly applies stress with baro receptor stimulating surface of the intraluminal device to stimulate the baro receptors at the cardiac portion of the stomach; and
wherein said second assembly comprises a plurality second assemblies with different stimulation levels, wherein amount of stress applied with the baro receptor stimulating surface to the cardiac portion of the stomach is selectable by replacing said second assembly with another second assembly having variation of at least one selected from (i) magnetic attraction strength, (ii) baro receptor stimulation contact area, (iii) baro receptor stimulation contact force, (iv) stiffness of the wall, (v) variation of thickness and/or distribution of material in the wall, (vi) weave of a structure wire, and (vii) material of the structure wire.

27. An intraluminal device that is adapted to apply stress to the cardiac portion of the stomach, comprising:
a magnetic fastener;
a wall defining a baro receptor stimulating surface, said baro receptor stimulating surface and the cardiac portion of the stomach conformable with each other;
said magnetic fastener having a magnet assembly and an attractor assembly, said magnet assembly generating magnetic flux and said attractor assembly being attracted to the magnet assembly in response to the magnetic flux, said magnet assembly comprising at least one electro-magnet; and
a first assembly comprising said magnet assembly or said attractor assembly, said first assembly configured to be fixed against the inner surface or an outer surface of the stomach at the cardiac portion of the stomach; and
a second assembly having said wall and the other of said one chosen from said magnet assembly and said attractor assembly, said second assembly adapted to be deployed to the stomach;
wherein the magnetic attraction between said magnet assembly and said attractor assembly applies stress with baro receptor stimulating surface of the intraluminal device to stimulate the baro receptors at the cardiac portion of the stomach; and
a controller adapted to adjust level of stimulation of the baro-receptors by controlling stress applied by the baro receptor stimulating surface to the cardiac portion of the stomach including adjusting amount of current to said at least one electro-magnet.
